(19) United States
(12) Patent Application Publication
(10) Pub. No.: US 2023/0148371 A1
(43) Pub. Date: May 11, 2023

(54) METHOD FOR DETECTING AND QUANTITATIVELY ASSESSING CARDIAC DYSSYNCHRONY

(71) Applicant: Region Nordjylland, Aalborg University Hospital, Aalborg Øst (DK)

(72) Inventor: Tomas Zaremba, Gistrup (DK)

(73) Assignee: Region Nordjylland, Aalborg University Hospital, Aalborg Øst (DK)

(21) Appl. No.: 18/046,196

(22) Filed: Oct. 13, 2022

Related U.S. Application Data
(63) Continuation of application No. 15/893,240, filed on Feb. 9, 2018, now abandoned.

(51) Int. Cl. A61B 5/02; A61B 5/11; A61B 8/08; A61B 5/055

(52) U.S. Cl. CPC A61B 5/02028; A61B 5/055; A61B 5/1107; A61B 8/488; A61B 8/0883; A61B 8/5223; A61B 8/5284

(57) ABSTRACT

The present disclosure relates to a method for detecting and/or quantitatively assessing cardiac dyssynchrony of a subject based on at least one medical imaging scan showing at least part of the myocardium of the subject’s heart, in particular mechanical cardiac dyssynchrony. The medical imaging scan may provide a plurality of values of a predefined myocardial deformation parameter of said part of the myocardium. In a preferred embodiment the method comprises the steps of 1) determining a myocardial deformation deviation between pairs of myocardial deformation parameter values selected from the myocardium of substantially opposite parts of a cardiac chamber, and 2) calculating the cardiac dyssynchrony of the subject based on said myocardial deformation deviation.

The present invention relates to a method for detecting cardiac dyssynchrony of a subject and quantitatively assessing the degree of cardiac dyssynchrony of said subject based on medical imaging, such as a 2D or 3D echocardiography, in particular mechanical cardiac dyssynchrony.

### BACKGROUND OF INVENTION

Cardiac dyssynchrony is a difference in the timing, or lack of synchrony, of contractions in different regions of the heart. It is caused by erratic electrical impulses that cause the chambers of the heart to pump out of sync. This limits the chambers from ejecting the blood as they normally would and causes the heart to work inefficiently. The condition is associated with patients who have been diagnosed with heart failure and is often caused by left bundle branch block (LBBB). Cardiac resynchronization therapy (CRT) is an established treatment in symptomatic systolic heart failure (HF) patients and the strongest body of evidence is seen in patients with LBBB in which activation of the left ventricle of the heart is delayed, which causes the left ventricle to contract later than the right ventricle. However, despite pre-selection by ECG morphology, the number of non-responders is still high. A more accurate method of detecting, characterizing and quantitatively assessing cardiac dyssynchrony can help improve identification of the condition and can indicate which treatment is best suited for the patient.

### SUMMARY OF INVENTION

The present disclosure therefore relates to a method for detection of cardiac dyssynchrony of a subject based on at least one medical imaging scan showing at least part of the myocardium of the subject’s heart. The medical imaging scan may provide a plurality of values of a predefined myocardial deformation parameter of said part of the myocardium. In a preferred embodiment the method comprises the steps of 1) determining a myocardial deformation deviation between pairs of myocardial deformation parameter values selected from the myocardium of substantially opposite parts of a cardiac chamber, and 2) calculating the cardiac dyssynchrony of the subject based on said myocardial deformation deviation. This method improves the detection of cardiac dyssynchrony, in particular the mechanical cardiac dyssynchrony, as it compares the deformation of different parts of the myocardium surrounding a cardiac chamber. This provides a more accurate evaluation of cardiac dyssynchrony, for example in comparison to prior art evaluation of electrical dyssynchrony and the treatment options. Today patients are typically screened for CRT based on LBBB, which is based on measuring electrical cardiac dyssynchrony. However, many patients which are not diagnosed with LBBB may suffer from mechanical cardiac dyssynchrony and may benefit from CRT. In that regard, the presently disclosed method is a major step forward in that patients can be identified which suffers from mechanical cardiac dyssynchrony but not necessarily LBBB.

The presently disclosed method can furthermore be applied for quantitatively assessing the degree of cardiac dyssynchrony, in particular the degree of mechanical cardiac dyssynchrony. This is a major advantage, for example for patients treated with Cardiac resynchronization therapy (CRT), because the quantitative assessment can be used for identifying responders following CRT and for predicting and evaluating the degree of response to CRT.

Hence, the present disclosure further relates to a method for assessing the degree of responsiveness of cardiac resynchronization therapy (CRT) of a subject based on quantitatively assessing cardiac dyssynchrony of said subject according methods disclosed herein. Correspondingly the present disclosure relates to a method for assessing the need for surgery, such as cardiac surgery, such as cardiac resynchronization therapy (CRT), of a subject based on detecting cardiac dyssynchrony of said subject according to the methods disclosed herein.

In one embodiment of the present invention the predefined myocardial deformation parameter is selected from the group; strain, strain rate, torsion, torsion rate, rotation, rotation rate, twist, twist rate, untwist and untwist rate. It may further be advantageous to include multiple parameters when determining the cardiac dyssynchrony of a subject. These parameters all relate to the deformation of the heart. Cardiac dyssynchrony is the result of the heart contracting asynchronously, meaning that these deformation parameters are well-suited for evaluating cardiac dyssynchrony. The method may be based on any kind of medical imaging scan that is able to show the contraction or deformation of the heart. In one embodiment the medical imaging scan is echocardiography, or speckle tracking echocardiography, or tissue Doppler echocardiography, or feature tracking cardiac magnetic resonance imaging, or tagging cardiac magnetic resonance imaging, or cardiovascular magnetic resonance imaging.

Echocardiography may be preferred in some embodiments as it is cost efficient method for measuring characteristics of the heart and it is available at many medical clinics.

### DESCRIPTION OF DRAWINGS

FIG. 1 shows an example of extraction of strain rate values from systolic curved anatomical M-mode plot obtained from a 3 chamber view. The color scale ranging from -1.8 s⁻¹ to +1.8 s⁻¹ provided in EchoPAC (r) used for decoding is shown in the upper-right corner.

FIG. 2 shows a flowchart of the calculation of strain rate mechanical dyssynchrony index for one embodiment of the disclosure. The two upper rows represent a four-chamber view, and the two lower rows represent an interpolated angle of 80 degrees outside the standard apical views.

FIG. 3 is a comparison of exported EchoPAC (r) systolic strain rate values and curved anatomical M-mode plot-based values in a three-chamber view representing the six traditional strain rate curves according to an embodiment of the disclosure.

FIG. 4 shows an example where responders exhibited significantly higher strain rate-based mechanical dyssynchrony index (SR-MDI) on pre-implantation echocardiograms than non-responders in three-chamber view and four-chamber view scans.

FIG. 5 is an example of measured baseline SR-MDI and reduction in SR-MDI around the left ventricle after cardiac resynchronization therapy. The greatest difference between the mean pre-implantation SR-MDI in responders and non-responders was seen at the 100-degree angle in the clockwise direction, which is located 10 degrees clockwise from the horizontal LV axis.

FIG. 6A shows one embodiment of the phases of left ventricular activation at pre-implantation echocardiography in a responder to cardiac resynchronization therapy.

FIG. 6B shows one embodiment of the phases of left ventricular activation at pre-implantation echocardiography in a non-responder to cardiac resynchronization therapy.

FIG. 7 shows an example of bull’s-eye plots of pre-implantation peak negative strain rate timing during systole in a CRT responder and a CRT non-responder. Both subjects had non-ischemic cardiomyopathy. The white dashed line shows the area of latest activation in the responder.

FIG. 8 shows examples of linear regression of SR-MDI and LV end-systolic volume. Upper row: baseline SR-MDI; lower row: reduction in SR-MDI after CRT.

FIG. 9 is an example of post-implantation predictors of positive response to cardiac resynchronization therapy.

FIG. 10 shows a Bland-Altman plot of SR-MDI of the three standard long-axis views according to one embodiment of the disclosure.

FIG. 11 shows an EchoPAC screenshot of the source image for an embodiment where the heart is scanned using echocardiography.

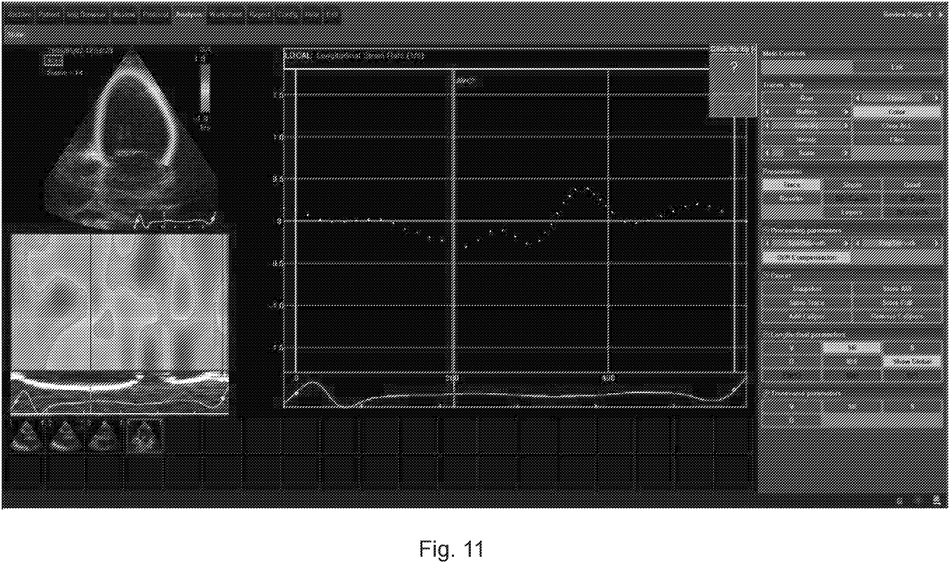

FIG. 12 is a zoom of the screenshot in FIG. 11 showing the strain rate in a curved anatomical m-mode plot.

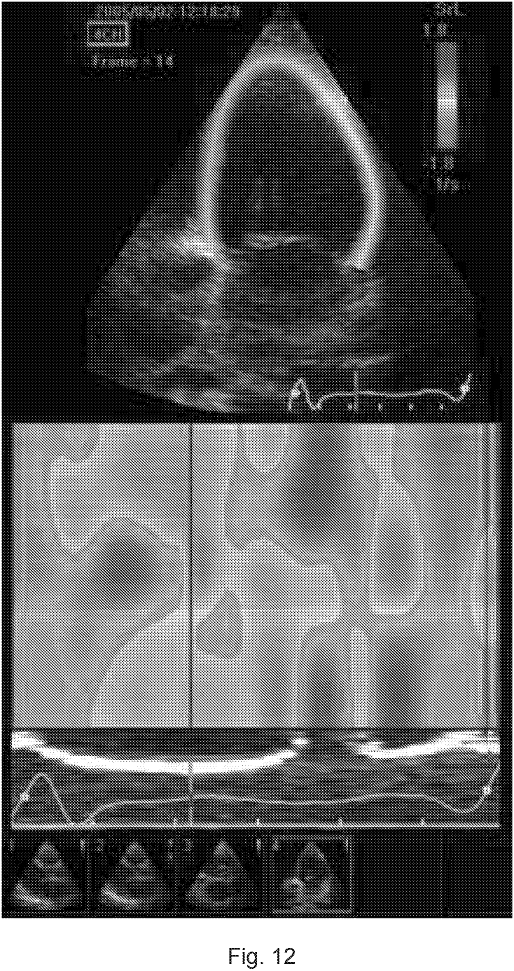

FIG. 13 is an example where the heart was scanned from three direction (2-chamber, 3-chamber and 4-chamber apical views) using echocardiography and showing the strain rate for the three directions. In this example the data are 200 points (corresponding to 200 frames during the time interval) and two 165-point measurements of each side of the chamber.

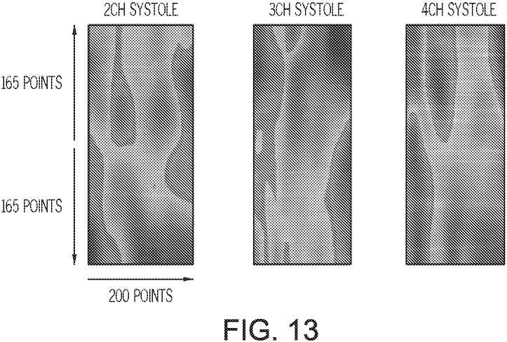

FIG. 14 shows one embodiment of the interpolation of data points from 2D strain rate data to form 3D strain rate data. For each frame, corresponding to a specific time in the cardiac cycle, pairs of points for the three scans are used to form data for six angles which is interpolated to form data with higher resolution.

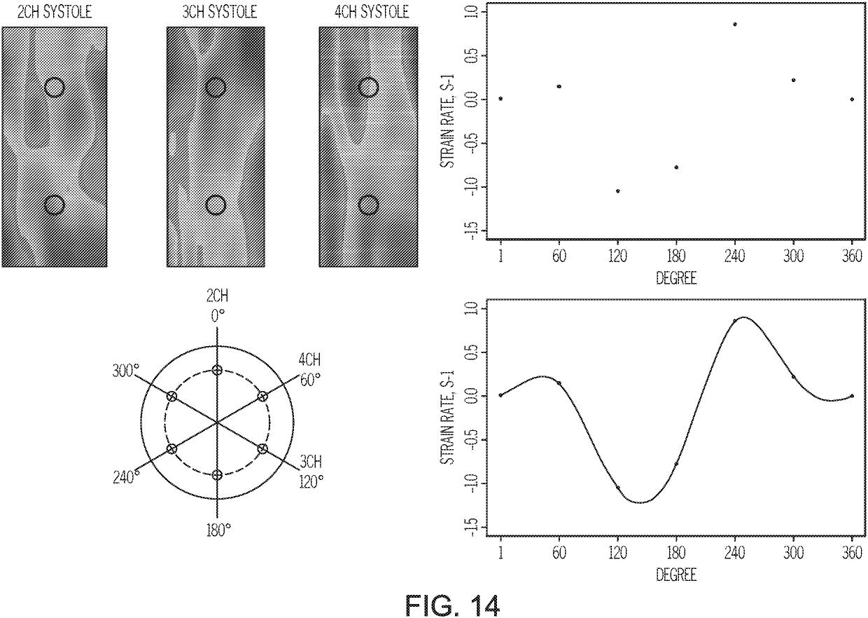

### DETAILED DESCRIPTION OF THE INVENTION

The present disclosure relates to a method for detecting and/or quantitatively assessing cardiac dyssynchrony in a subject’s heart. The method is based on a plurality of values from at least one medical imaging scan which is used to calculate a myocardial deformation deviation. In one embodiment the medical imaging scan is part of the claimed method. In one embodiment the myocardial deformation deviation is calculated as the difference between pairs of myocardial deformation parameter values. The pairs preferably belong to regions of the cardiac wall of a cardiac chamber that are substantially opposite of each other, e.g. opposing walls of the cardiac chamber, such that one value from one side of the chamber is subtracted from another value at substantially the other side of the chamber. A difference in timing between contractions at different regions of the heart may thereby be detected from the myocardial deformation deviation. As an example, the strain rate may be used as an indicator of dyssynchrony in which case the myocardial deformation deviation should be low for synchronous contractions of the heart.

In one embodiment of the disclosure the cardiac chamber, from which the myocardial deformation parameter values are obtained, is the right atrium, more preferably the left atrium, even more preferably the right ventricle, most preferably the left ventricle. The detection of cardiac dyssynchrony may be based on medical imaging scans of any of the four cardiac chambers. It may be most advantageous to analyse the left ventricle, as this is the strongest chamber pumping blood to the entire body. In one embodiment the at least one medical imaging scan shows the entire left ventricle of the subject’s heart. In another embodiment the at least one medical imaging scan is an apical view, such as an apical 4 chamber view, or an apical 3 chamber view, or an apical 2 chamber view.

Medical imaging techniques used to scan the heart may provide a 2D image of the heart showing a single plane through the heart. As an example, this can be obtained using standard echocardiography. In one embodiment the method is based on myocardial deformation parameter values from one or more medical imaging scans showing a single plane of the heart. In another embodiment the method is based on myocardial deformation parameter values from medical imaging scans showing multiple planes of the heart, such as at least 2 planes, or at least 3 planes, or at least 4 planes. Imaging multiple planes may be advantageous, as this shows more parts of the heart and therefore may provide a more accurate analysis.

In an embodiment of the present disclosure the at least one medical imaging scan is obtained at a preselected time of the cardiac cycle, such as the onset of the systole, the mid-systole, the end-systole, the start-diastole, the mid-diastole, or the end-diastole. This preselected time of the cardiac cycle could advantageously be during the systolic part where the heart is contracting and the myocardial deformation parameter could show large values. The method is in another embodiment based on multiple medical imaging scans during a preselected part of the cardiac cycle, such as the systolic part of the cardiac cycle or the diastolic part of the cardiac cycle. In a further embodiment the medical imaging scans are obtained continuously for at least a selected part of the cardiac cycle, such as the systolic part of the cardiac cycle. Continuously imaging part of the cardiac cycle may be advantageous as it provides more data and thereby can make the analysis more thorough.

The method of the present disclosure is based on at least one medical imaging scan which may be a 2D scan or a 3D scan. This means that the medical imaging device, such as equipment for obtaining an echocardiogram, may provide a 2D image of at least part of the heart or a 3D image of at least part of the heart. In one embodiment 2D medical imaging scans showing multiple views of the heart are used to calculate at least one 3D image by interpolating the 2D scans, wherein said at least one 3D image provides a plurality of values of the myocardial deformation parameter for at least part of the myocardium which is used for calculating the cardiac dyssynchrony of the subject. The 2D medical imaging scans may be acquired at selected angles or at angles with regular intervals, for example 3 planes with 120 degrees separation, such that the interpolation is carried out on six sets of data separated by 60 degrees around an axis passing through the chamber and the apex of the heart.

The interpolation technique used to calculate the at least one 3D image may be linear interpolation, polynomial interpolation, spline interpolation, cubic interpolation, bilinear interpolation, bicubic interpolation, trigonometric interpolation or inverse distance weighted interpolation. The interpolation technique may also be any of the mentioned techniques applied in 2D and/or applied to a periodic function. The myocardial deformation parameter values may be provided along parts of the cardiac chamber wall, and may therefore in some embodiments be viewed as a two-dimensional cyclic set of data points. This data may in one embodiment be considered as cyclic around an axis passing through the chamber and pointing to the apex of the heart.

Instead of calculating a 3D image from the medical imaging scans, the provided myocardial deformation parameter values from the medical imaging scans may also be used for the interpolation. Therefore, in another embodiment, 2D myocardial deformation parameter values from multiple views of the heart are used to calculate 3D myocardial deformation parameter values of at least part of the myocardium by interpolating the 2D myocardial deformation parameter values such that said 3D myocardial deformation parameter values are used for calculating the cardiac dyssynchrony of the subject. The interpolation may be performed using any of the techniques described previously.

In some embodiments it may be preferred to continuously acquire data from multiple directions each covering at least part of the systolic part of the cardiac cycle such that the data may be interpolated over this time interval. Therefore, in another embodiment the plurality of myocardial deformation parameter values are provided by continuously scanning at least part of the myocardium in 2D from multiple directions over at least part of the systolic part of the cardiac cycle, and wherein said 2D myocardial deformation parameter values are interpolated to form 3D myocardial deformation parameter values used for calculating the cardiac dyssynchrony of the subject. The data acquired from multiple directions may be acquired simultaneously or individually. The data does not necessarily need to be acquired at the same time, i.e. they do not need to represent the same cardiac cycle. For embodiments where the data is acquired individually for each direction, the data can be synchronized to the cardiac cycle of data from the other directions. Synchronizing this data may also be performed using electrocardiograms recorded together with the medical imaging scans.

In one embodiment of the present invention, the at least one medical imaging scan provides a plurality of myocardial deformation parameter values along the cardiac chamber wall of the left ventricle. In another embodiment the at least one medical imaging scan provides a plurality of myocardial deformation parameter values covering the interventricular septum and the lateral wall of the myocardium. This may be preferred for detecting cardiac dyssynchrony, as the lateral wall is an outer part of the heart and the interventricular septum is a central part of the heart located between the left and right ventricle, such that dyssynchrony may be more evident from analysing data from these regions. For such embodiments the substantially opposite parts of the cardiac chamber are the interventricular septum and the lateral wall myocardium. The inventor has realized that using many myocardial deformation values provides a better measure of the cardiac dyssynchrony, preferably at least at least 25 or at least 50, more preferably at least 100, even more preferably at least 200, most preferably at least 300 myocardial deformation deviation values covering a substantial part of one of the cardiac chambers.

In a specific embodiment of the disclosure, the myocardial deformation deviation is calculated by mirroring the first half of the plurality of myocardial deformation parameter values and subtracting this from the second half of the plurality of myocardial deformation parameter values, and wherein said first and second halves of the plurality of myocardial deformation parameter values correspond to substantially opposite parts of the cardiac chamber. The medical imaging scan may provide myocardial deformation parameter values along the lateral wall, past the apex of the heart and along the interventricular septum. For such scans the mirroring method may be advantageous, as a given value is then subtracted from the corresponding point on the opposite side of the chamber. This forms pairs of data starting from the apex of the heart and going to the base of the chamber.

The data obtained from the medical imaging scans are used for determining the cardiac dyssynchrony of the subject. The data may be from a single scan, multiple scans of the same area, continuous scans over a time interval, 3D data from interpolating 2D scans, 3D data from interpolating 2D scans over a time interval, a single 3D scan or 3D scans over a time interval. The data is used to calculate the myocardial deformation deviation by calculating the difference between pairs of values from the data. In a specific embodiment, data is recorded in different directions over the systolic part of the cardiac cycle, the data is synchronized to the other scans, interpolated to form a 3D representation of the data which is then used to calculate the myocardial deformation deviation for all pairs of values in the data.

The data may be analysed in various ways to determine cardiac dyssynchrony of the subject. In one embodiment of the invention a plurality of values for the myocardial deformation deviation are used to calculate a histogram. In another embodiment the cardiac dyssynchrony of the subject is a statistical parameter selected from the group; standard deviation, variance, mean, harmonic mean, median, mode, range, quantile and maximum value of said histogram, or wherein said the cardiac dyssynchrony is based on one or more of said statistical parameters. The cardiac dyssynchrony may in some embodiments be characterised by the value of one or more of the statistical parameters. These one or more statistical parameters may form one or more dyssynchrony indices for evaluating cardiac dyssynchrony of the subject. In yet another embodiment the cardiac dyssynchrony is based on two or more of the statistical parameters, for example by combining two or more of the statistical parameters to form a dyssynchrony index for determining the cardiac dyssynchrony of the subject.

The dyssynchrony index for determining cardiac dyssynchrony of a subject may in some embodiments also be used to determine the degree of cardiac dyssynchrony from the value of the index. This could also be useful for improving identifying the condition of cardiac dyssynchrony. One or more of the calculated statistical parameters or dyssynchrony indices may also be useful for selecting the best treatment for the subject. This could be from the value of one or more parameters and/or indices, or from an analysis of one or more of the parameters and/or indices.

The presently disclosed method may in one embodiment be used for determining if a patient is suffering from cardiac dyssynchrony or not. In another embodiment the method is used to determine the degree of cardiac dyssynchrony of the subject. Thereby, the cardiac dyssynchrony is determined quantitatively. The quantitative measurement of cardiac dyssynchrony may be given by a single statistical parameter related to the cardiac deformation deviation values as described earlier. The quantitative measurement may also be calculated from two or more of the statistical parameters. This quantitative measurement of cardiac dyssynchrony may be used to evaluate the degree of cardiac dyssynchrony of the subject, the need for surgery such as cardiac resynchronization treatment, and for selecting the most suitable treatment for the subject and the degree of responsiveness to CRT.

The presently disclosed method may be implemented as a separate computer program but may also be implemented as a plugin to an existing service running on a device or online.

### System and Use

The present disclosure is further related to a system for detection of cardiac dyssynchrony of a subject based on at least one medical imaging scan showing at least part of the myocardium of the subject’s heart. The medical imaging scan provides a plurality of values of a predefined myocardial deformation parameter of said part of the myocardium. The system may accordingly be configured to carry out the methods disclosed herein. The system may further comprise a non-transitive, computer-readable storage device for storing instructions that, when executed by a processor, performs a method for detection of cardiac dyssynchrony of a subject according to the methods disclosed herein. The system may further comprise a device comprising a processor and a memory and being adapted to perform the methods. It can also be a system operating from a centralized location, and/or a remote system, involving e.g. cloud computing.

The present disclosure further relates to a system for detecting and/or quantitatively assessing cardiac dyssynchrony of a subject, comprising a medical imaging device configured for acquiring a medical imaging scan of at least part of the myocardium of the subject’s heart, said medical imaging scan providing a plurality of values of a predefined myocardial deformation parameter of said part of the myocardium, a processing unit for carrying out the method for detecting and/or quantitatively assessing cardiac dyssynchrony of a subject according to method disclosed herein.

The presently disclosed methods may be fully or partly computer implemented.

The present disclosure further relates to a computer program having instructions which when executed by a computing device or system causes the computing device or system to detect cardiac dyssynchrony of a subject according to the described method. Computer program in this context shall be construed broadly and include e.g. programs to be run on a PC or software designed to run on smartphones, tablet computers or other devices. Computer programs and applications include software that is free and software that has to be bought.

### EXAMPLE - MECHANICAL DYSSYNCHRONY INDEX BASED ON SYSTOLIC STRAIN RATE

### Study Population

The study was conducted as an analysis of prospectively acquired data in patients with HF having LBBB who underwent implantation of CRT device at two centers: Aalborg University Hospital and Gentofte University Hospital. Only patients fulfilling ECG criteria for native LBBB and QRS duration ≥ 120 ms were included (6). Follow-up period was six months after the CRT implantation. Positive response to CRT was defined as a reduction of LV end-systolic volume (ESV) by ≥ 15% compared to baseline echocardiography. In total, 101 patients were included (Aalborg n=58, Gentofte n=43). In addition, echocardiograms from 10 healthy persons were used as controls. The study protocol was approved by the Institutional Review Boards at both centers and complied with the Declaration of Helsinki.

### Image Analysis

Strain rate analysis was performed on ECG-gated long-axis 2D-echocardiography images using EchoPAC (r) software version 201 (GE Healthcare, Milwaukee, WI). Two-chamber, three-chamber, and four-chamber 2D images acquired at mean frame rate of 66.6 ± 11.1 s⁻¹ were analyzed. The duration of systole was defined as the period from the onset of QRS complex to the aortic valve closure in trans-aortic continuous wave Doppler plot. Afterwards, a regular STE-based strain rate analysis was performed. Standard echocardiography analysis packages such as EchoPAC (r) usually export quantitative data of six segments per view. A strain rate CAMM plot is a rectangular image depicting a strain rate value in each pixel. CAMM plots which are used for visual representation of strain rate propagation typically contain 330 data-lines per view (the vertical dimension of the CAMM plot).

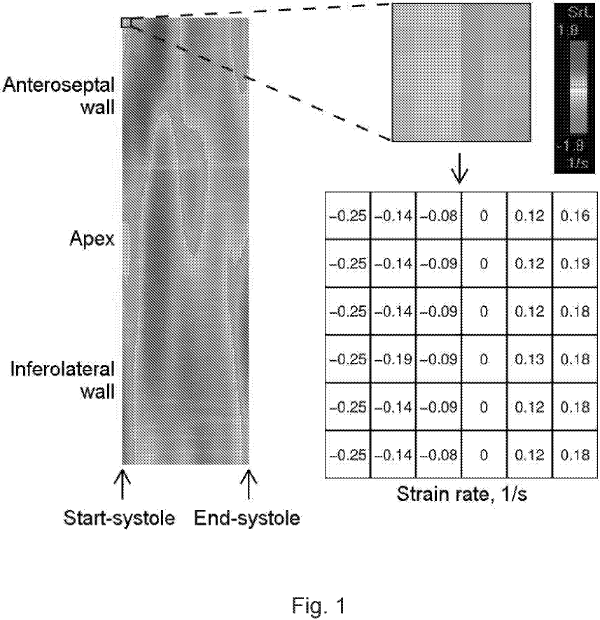

The first step of the present approach was to extract the strain rate data from CAMM plots. Each of the pixels of the systolic part of a CAMM plot was converted to a strain rate value. For this decoding, the color scale provided in the upper part of EchoPAC (r) display was used (FIG. 1). The resulting individual strain rate values from each pixel were then arranged in the same order as the corresponding pixels in the CAMM plot. This gives a table of strain rate values, which has the same number of rows and columns as the vertical and horizontal dimensions of the systolic CAMM plot. As in the original CAMM plot, the upper and the lower halves of the resulting table represent the strain rate from the two opposing walls of the LV, while the LV apex is “located” between the two halves. Anatomically basal parts of the LV seen in the particular apical projection are represented symmetrically in the top and in the bottom, both of the CAMM plot and likewise in the acquired table. The start-systole is on the left-hand side, and the end-systole is on the right-hand side of the table.

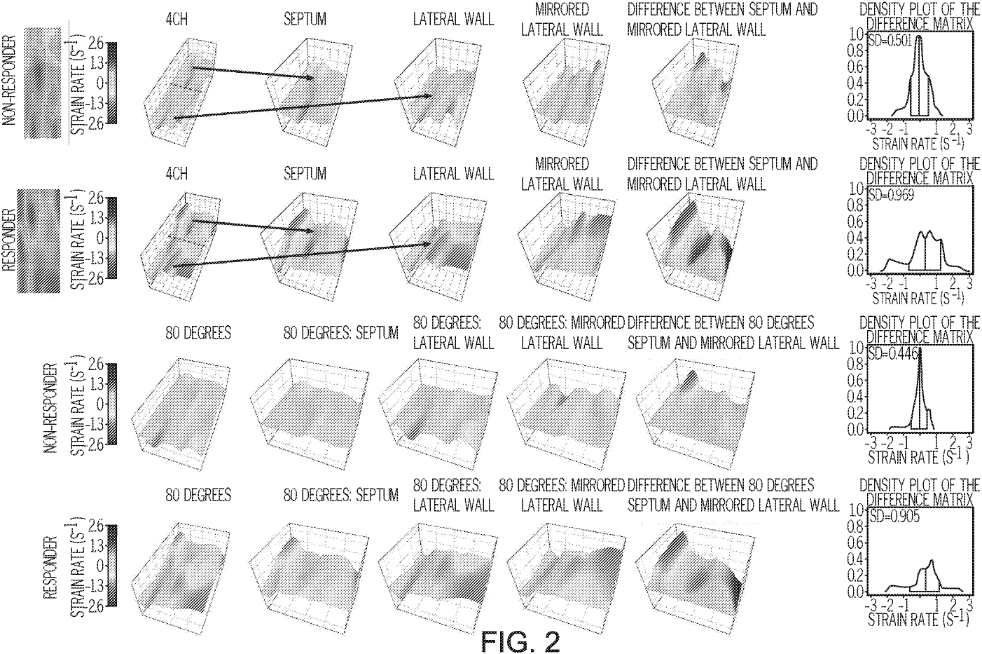

The next step was to construct a 3D model of strain rate throughout the systole. Data from the above tables of the three standard apical projections were used. Looking at the LV from the apex and setting the anterior portion of the LV to 0 degrees (12 o’clock), two-chamber, four-chamber, and three-chamber views cover the radii of 0-180, 60-240, and 120-300 degrees, respectively. Besides providing the instant strain rate data of these six LV walls, the strain rate data from the three CAMM-based tables contain the strain rate values in the three apical cross-sections throughout the systole. Meanwhile, no data are available for other LV cross-sections such as for 10-190 degrees-slice. To calculate the strain rate values in all the rest of 360 degrees (radii) of the LV, cubic spline interpolation was performed. This was based on the known values of strain rate contained in the three standard projections. An R (r) package ‘stats’ version 3.2.4 was used, and the interpolation was performed individually for all circles along LV from the apex to the base. An analogy with a globe could be made, where the data points located along six meridians (lines of longitude) are available, and the interpolation was then performed along the parallels (lines of latitude) to generate the remaining 354 meridians. Likewise, the same procedure was performed for every phase of the systole. Thus, strain rate data in the remaining of 360 degrees covering the whole LV during entire systole were calculated. A total of 180 apical cross-sections were generated, each represented by an analogous table containing strain rate values. The mechanical LV dyssynchrony was then assessed by comparing the systolic strain rate properties of opposing LV walls (FIG. 2). First, the analysis was performed in the three strain rate tables of the initially obtained standard views. In matrix algebra, subtraction of two tables (matrices) is performed by element wise subtraction of corresponding elements, which results in a new matrix of the same dimensions. Hence, the two geometrically opposing upper and lower halves of the strain rate table were subtracted from each other. Here, all symmetrical table entries of the opposing LV walls were subtracted one by one. This provided a new table containing the differences in systolic strain rate in the two opposing walls based on the table from each particular cross-section.

In case of an almost symmetric LV contraction, such resulting table would contain uniform values relatively close to zero. On the other hand, an asymmetric contraction would result in greater strain rate differences of the two opposing walls. In other words, contraction in one wall (negative strain rate value) and simultaneous stretch in opposite wall (positive strain rate value) would lead to a high absolute value when these symmetrical numbers are subtracted from each other. Standard deviation (SD) of all such strain rate differences in the CAMM-plot-derived difference table was then used as a measure to quantify the dispersion of the strain rate differences in the two opposing walls [strain rate-based mechanical dyssynchrony index (SR-MDI)] in the particular cross-section (FIG. 2).

Besides the standard apical views, the same quantification of SR-MDI was performed separately for all 180 tables of strain rate values obtained through the cubic spline interpolation procedure. SR-MDI values from all 180 cross-sections per patient were used as predictor variables for positive CRT response. The cross-section, having the highest area under a receiver operating characteristic (ROC) curve for SR-MDI as CRT response predictor, was used in further analysis along with SR-MDI of the three standard apical views.

The corresponding strain rate analysis was performed on post-CRT echocardiograms at least 6 months after CRT. Also, the SR-MDI quantification was repeated in a blinded fashion in a random sample of 10 pre-implantation echocardiograms to determine the degree of intra-observer agreement. The repeated analyses were performed in all three standard projections, thus rendering a sample of 30 repeated observations. Intraclass correlation coefficient, coefficient of variability, and bias were calculated.

LV EF and LV ESV were calculated using biplane Simpson’s method by blinded experienced observer using EchoPAC (r) software. The analyses were performed on pre-implantation 2D echocardiography images and on follow-up echocardiography images obtained at least 6 months after the implantation.

### Clinical Characteristics

Medical records of the patients were reviewed manually. Following pre-implantation data were collected: New York Heart Association class, anticongestive and lipid-lowering medical therapy, renal function, and QRS duration on the 12-lead ECG. Chronic kidney disease was defined as estimated glomerular filtration rate below 60 ml/min/1.73 m² body surface area. Ischemic etiology of cardiomyopathy was defined as patients having previous diagnosis of acute coronary event or underwent a revascularization procedure, or had a significant coronary artery stenosis (>70%).

### Statistical Analysis

Continuous variables were reported as mean along with their SD. Categorical values were reported as absolute numbers and percentages. Continuous variables were compared by two-sample Student t-test. Fisher exact test was used to compare categorical variables. Linear regression and Pearson’s r were applied to evaluate association between two linear variables. Logarithmic transformation of data was used in case it improved the fit of the model. ROC analysis was used to identify the cutoff and predictive values of SR-MDI and QRS duration. Univariate logistic regression was used to evaluate predictor values selected from clinical perspective in advance. Afterwards, multivariate logistic regression model was built. Two-sided tests were used, and p<0.05 was considered statistically significant. All analyses were performed on R (r) version 3.2.4.

### Results

Demographic and baseline characteristics stratified by CRT response are provided in Table 1. According to defined criteria of LV ESV reduction by ≥ 15% after 6 months, 74 (73.3%) subjects out of 101 responded to CRT. Mean age was 68 ± 9 years, and 37 (36.6%) were female. Mean pre-implantation LV EF was 26.5 ± 7.1% and mean QRS duration was 162 ± 21 ms. Responders were younger and had a lower prevalence of chronic kidney disease compared to non-responders.

TABLE 1

Baseline characteristics

| | All subjects (n=101) | Responders (n=74) | Non-responders (n=27) | p-value |
|---|---|---|---|---|
| Age, yrs | 68 ± 9 | 66 ± 9 | 71 + 8 | 0.01* |
| Female, n (%) | 37 (36.6) | 31 (41.9) | 6 (22.2) | 0.1 |
| Ischemic etiology, n (%) | 63 (62) | 43 (58.1) | 20 (74.1) | 0.1 |
| NYHA-class | | | | 0.94 |
| I, n (%) | 1 (1) | 1 (1.4) | 0(0) | |
| II, n (%) | 37 (36.6) | 28 (37.8) | 9 (33.3) | |
| III, n (%) | 63 (62.4) | 46 (62.2) | 17 (63) | |
| ACEI/ARB, n (%) | 98 (97) | 72 (97.3) | 26 (96.3) | 0.99 |
| Beta-blockers, n (%) | 96 (95) | 70 (94.6) | 26 (96.3) | 0.99 |
| Loop diuretics, n (%) | 69 (68.3) | 47 (63.5) | 22 (81.5) | 0.1 |
| Aldosterone antagonists, n (%) | 57 (56.4) | 41 (55.4) | 16 (59.3) | 0.82 |
| Statins, n (%) | 72 (71.3) | 50 (67.6) | 22 (81.5) | 0.22 |
| eGFR <60 ml/min/1.73 m², n (%) | 46 (45.5) | 29 (39.2) | 17 (63) | 0.04* |
| LV ESV, ml | 135.5 ± 52.4 | 136.1 ± 56.2 | 133.8 ± 41.2 | 0.83 |
| LV EF, % | 26.5 ± 7.1 | 26.7 ± 6.9 | 26.1 ± 7.7 | 0.75 |
| QRS duration, ms | 162 ± 21 | 162 ± 19 | 162 ± 26 | 0.97 |

ACEI = angiotensin-converting enzyme inhibitors; ARB = angiotensin II receptor blockers; EF = ejection fraction; eGFR = estimated glomerular filtration rate; ESV = end-systolic volume; LV = left ventricle; NYHA = New York Heart Association; * = p<0.05.

### Pre-Implantation SR-MDI

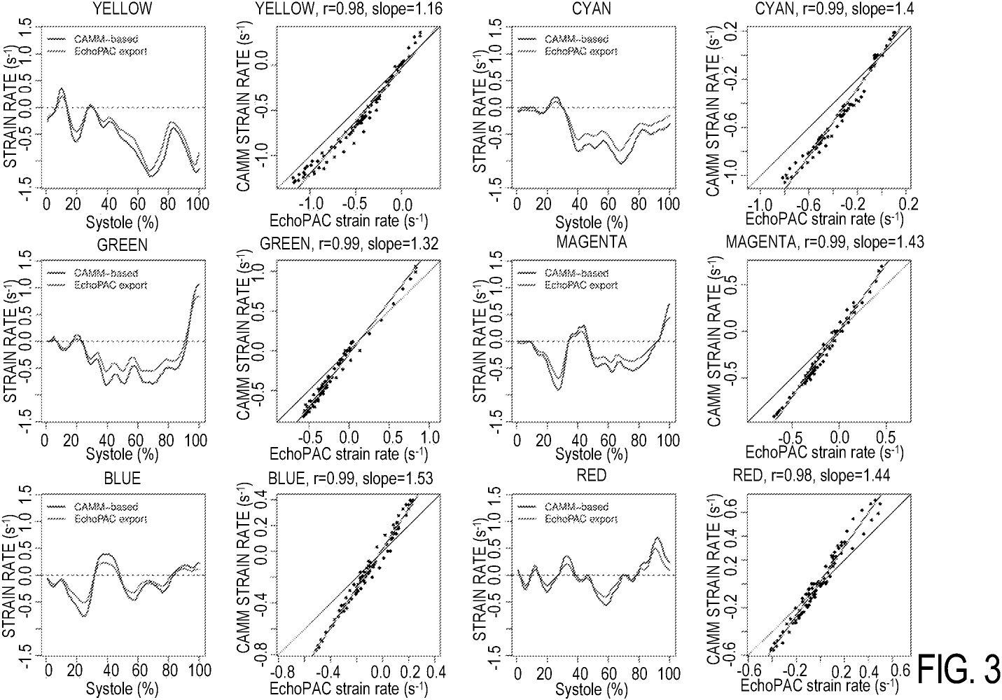

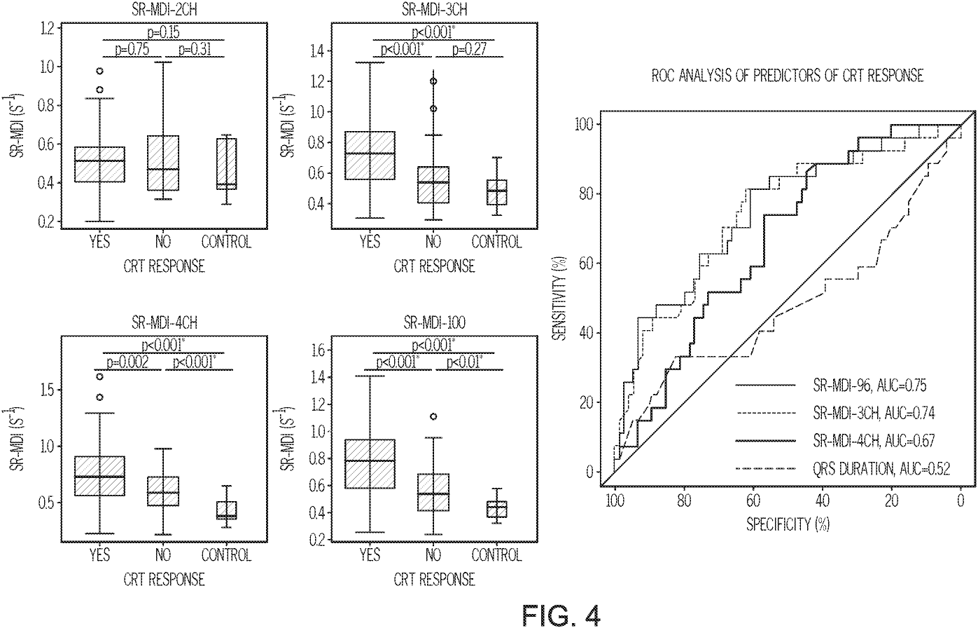

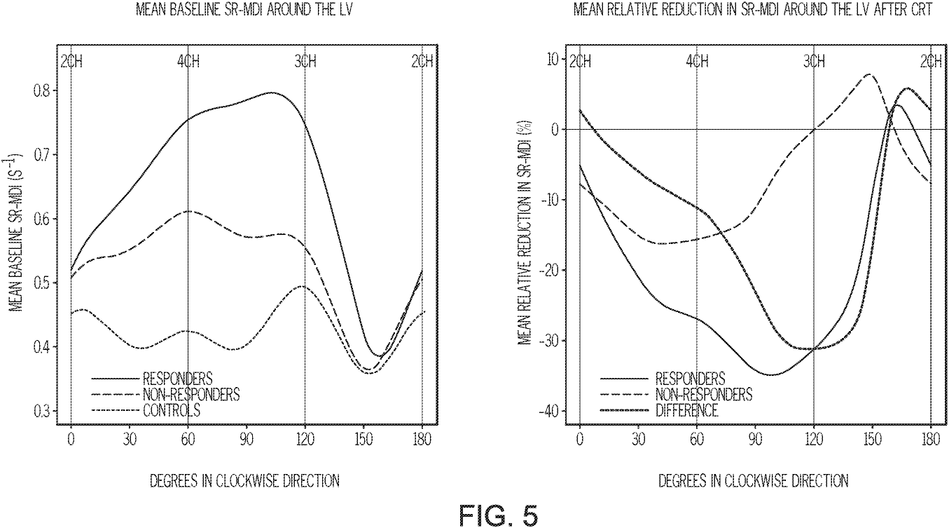

An example of traditionally exported strain rate curves from EchoPAC (r) and CAMM plot-generated curves at the identical rows in the CAMM plot are shown in FIG. 3. Responders exhibited significantly higher SR-MDI on pre-implantation echocardiograms than non-responders in three-chamber view (SR-MDI-3ch) (0.74 ± 0.25 s⁻¹ vs. 0.55 ± 0.22 s⁻¹, p<0.001) and four-chamber view (SR-MDI-4ch) (0.75 ± 0.26 s⁻¹ vs. 0.61 ± 0.18 s⁻¹, p=0.002) (FIG. 4). No significant difference in SR-MDI was found between responders and non-responders in two-chamber view (SR-MDI-2ch) (p=0.75). The greatest difference between the mean pre-implantation SR-MDI in responders and non-responders was seen at the 100-degree angle in the clockwise direction (0.8 ± 0.26 s⁻¹ vs. 0.57 ± 0.22 s⁻¹, p<0.0001), which is located 10 degrees clockwise from the horizontal LV axis (FIG. 5).

Compared to controls, non-responders had higher SR-MDI-4ch, while responders had higher SR-MDI-3ch and SR-MDI-4ch. QRS duration did not differ between the two groups undergoing CRT (p=0.97).

ROC analysis of SR-MDI-3ch for prediction of CRT response yielded an AUC of 0.74 [95% confidence interval (Cl) 0.63-0.86] which was higher than AUC of QRS duration of 0.52 (95% Cl 0.37-0.66), p=0.01 (FIG. 4). Threshold of SR-MDI-3ch at 0.66 s⁻¹ had sensitivity 62.2%, specificity 81.5%, positive predictive value (PPV) 90.2%, negative predictive value (NPV) 44%, and accuracy 67.3%. ROC analysis of SR-MDI-4ch for prediction of CRT response had an AUC of 0.67 (95% Cl 0.56-0.78) which was not significantly higher than AUC of QRS duration (p=0.09). Threshold of SR-MDI-4ch at 0.7 s⁻¹ had sensitivity 56.8%, specificity 74.1%, PPV 85.7%, NPV 38.5%, and accuracy 61.4%.

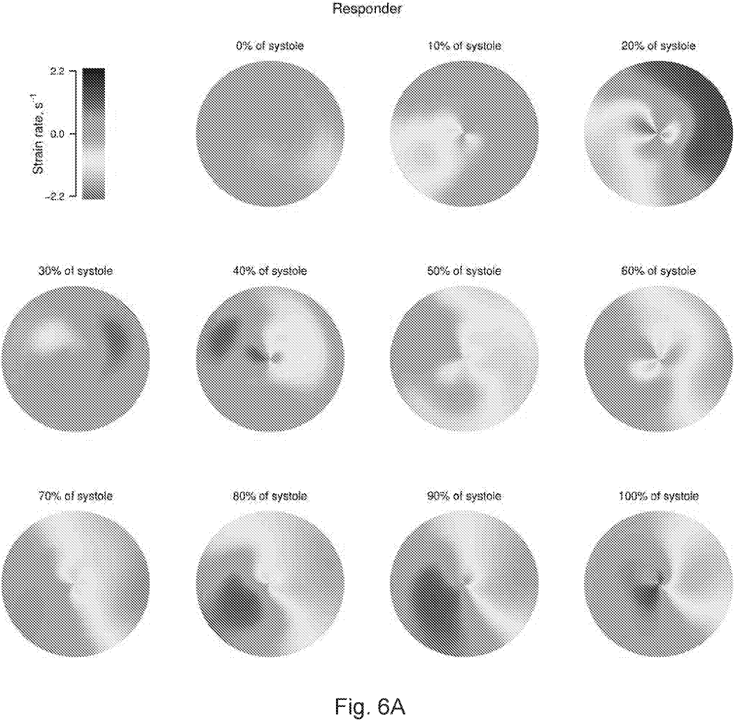

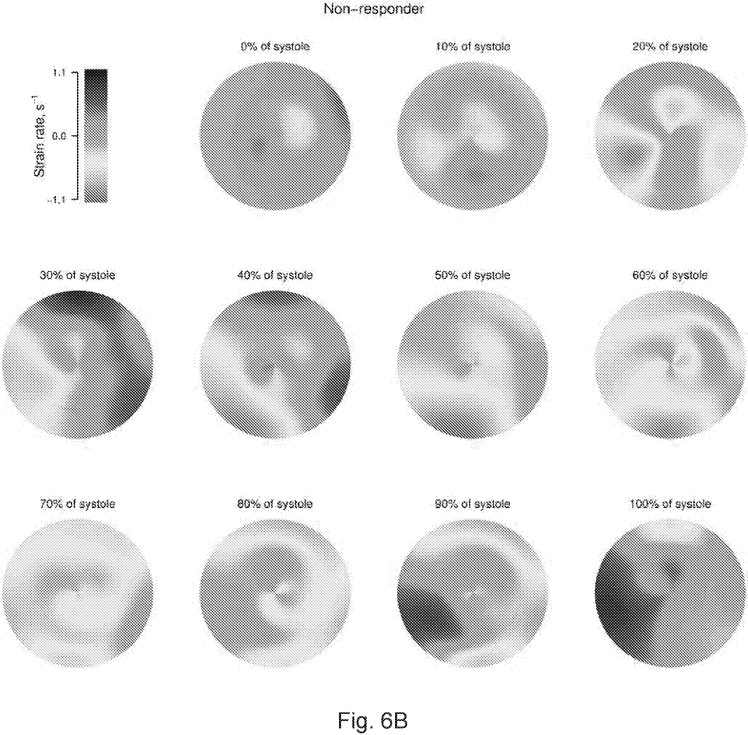

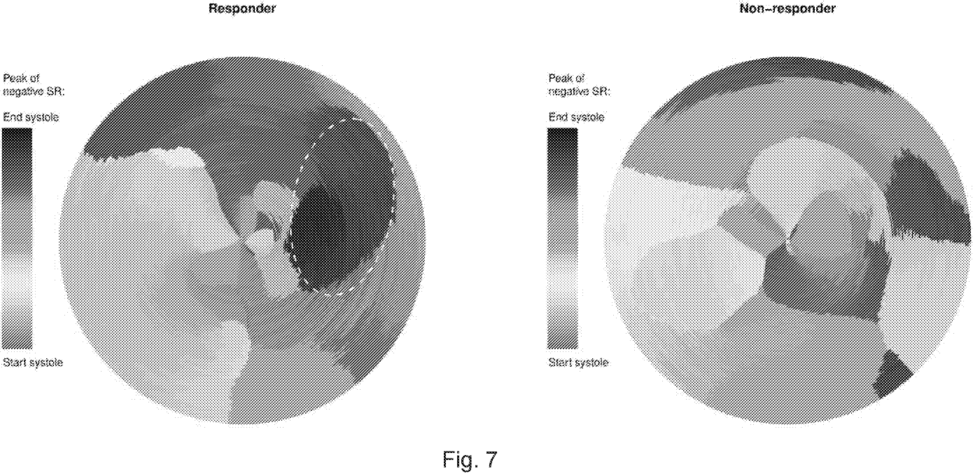

Separate ROC analyses covering 180 different axes of LV showed that the axis with the highest predictive value for CRT response based on AUC transected LV at a 96-degree angle in the clockwise direction. SR-MDI at this angle (SR-MDI-96) had an AUC of 0.75 (95% Cl 0.64-0.86) which was higher than AUC of QRS duration (p=0.009). Threshold of SR-MDI-96 at 0.72 s⁻¹ (fulfilled in 49.5% cases) had sensitivity 60.8%, specificity 81.5%, PPV 90%, NPV 43.1%, and accuracy 66.3%. In addition, the reconstruction of 180 radii of strain rate throughout the systole enabled a visual representation of LV activation from a polar plot perspective (FIGS. 6A and 6B) and timing of peak negative strain rate during systole (FIG. 7).

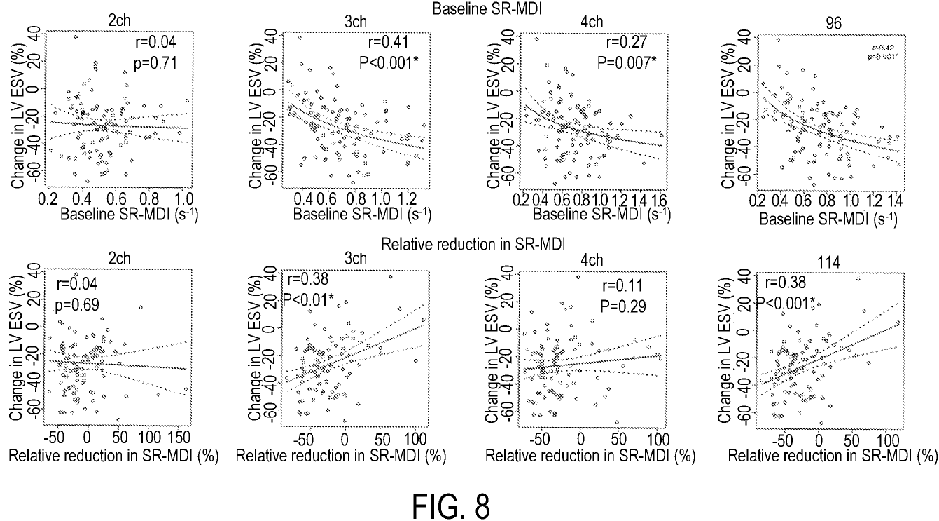

A linear relationship between the degree of LV ESV reduction after CRT and the pre-implantation SR-MDI was observed as well (FIG. 8). Statistically significant association was found in SR-MDI-3ch (r=0.41, p<0.0001), SR-MDI-4ch (r=0.27, p=0.007), and SR-MDI-96 (r=0.42, p<0.0001), but not in SR-MDI-2ch.

The results of the logistic regression are summarized in Table 2. SR-MDI values were logtransformed using logarithm to base 2. Odds ratio in such case represents an odds ratio associated with a doubling of the independent variable. In a univariate model, CRT response was predicted by SR-MDI-3ch, SR-MDI-4ch, SR-MDI-96, age, and renal function. The multivariate models contained age, renal function and SR-MDI in 3ch, 4ch, and at 96 degrees as predictors. SR-MDI-3ch, SR-MDI-4ch, and SR-MDI-96 were analyzed separately due to collinearity. After correction for age and renal function, SR-MDI-3ch, SR-MDI-4ch, and SR-MDI-96 were found to independently predict CRT response.

TABLE 2

Logistic regression of predictors of response to cardiac resynchronization therapy

| | Odds ratio (95% Cl) | p-value |
|---|---|---|
| Univariate analysis | | |
| Age | 0.94 (0.89-0.99) | 0.02* |
| Male sex | 0.4 (0.13-1.05) | 0.07 |
| eGFR <60 ml/min/1.73 m² | 0.38 (0.15-0.93) | 0.04* |
| QRS duration | 1 (0.98-1.02) | 0.96 |
| Ischemic etiology | 0.49 (0.17-1.25) | 0.15 |
| Log₂ SR-MDI-2ch | 1.38 (0.49-3.99) | 0.54 |
| Log₂ SR-MDI-3ch | 6.12 (2.36-18.2) | <0.001* |
| Log₂ SR-MDI-4ch | 2.89 (1.22-7.39) | 0.02* |
| Log₂ SR-MDI-96 | 5.93 (2.39-16.9) | <0.001* |
| Multivariate model 1 | | |
| Age | 0.93 (0.87-0.99) | 0.03* |
| eGFR <60 ml/min/1.73 m² | 0.51 (0.18-1.41) | 0.2 |
| Log₂ SR-MDI-3ch | 7.06 (2.53-23.1) | <0.001* |
| Multivariate model 2 | | |
| Age | 0.94 (0.89-1) | 0.05 |
| eGFR <60 ml/min/1.73 m² | 0.54 (0.2-1.42) | 0.21 |
| Log₂ SR-MDI-4ch | 2.73 (1.09-7.46) | 0.04* |
| Multivariate model 3 | | |
| Age | 0.93 (0.87-0.99) | 0.03* |
| eGFR <60 ml/min/1.73 m² | 0.55 (0.19-1.53) | 0.26 |
| Log₂ SR-MDI-96 | 6.61 (2.47-20.8) | <0.001* |

2ch = two-chamber view; 3ch = three-chamber view; 4ch = four-chamber view; 96 = 96-degree angle; eGFR = estimated glomerular filtration rate; Log₂ = binary logarithm;
SR-MDI = strain rate myocardial dyssynchrony index; * = p<0.05.

### Reduction in SR-MDI After CRT

Post-CRT echocardiograms at 6 months were available for STE analysis in 94 (93.1%) patients (69 responders, 25 non-responders). The reduction in SR-MDI was significantly greater in responders than in non-responders in three-chamber view (-31.36 ± 30.42% vs. -0.23 ± 43.03%, p=0.002). The greatest difference between mean reduction in SR-MDI was seen in a 117-degree angle (-31.99 ± 29.79% vs. -0.85 ± 42.92%, p=0.002). No significant difference in SR-MDI improvement after CRT was found between the two groups in two- and four-chamber views.

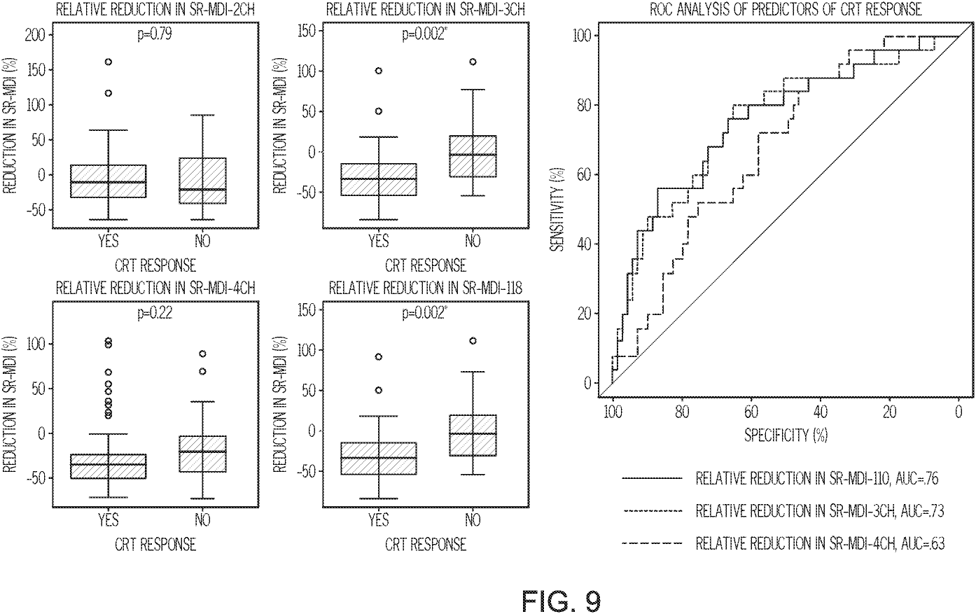

In ROC analysis, the reduction in SR-MDI-3ch after implantation was associated with CRT response providing an AUC 0.73 (95% Cl 0.61-0.85). The highest AUC [0.76 (95% Cl 0.64-0.87)] of SR-MDI reduction as predictor of CRT response was found in a 110-degree angle clockwise (FIG. 9).

There was a linear correlation between the reduction in LV ESV and improvement in SR-MDI in three-chamber view and in the 110-degree angle (r=0.38, p<0.001 and r=0.38, p<0.001, respectively). No significant correlation was found in the two- and four-chamber views (FIG. 8).

### Intraobserver Analysis

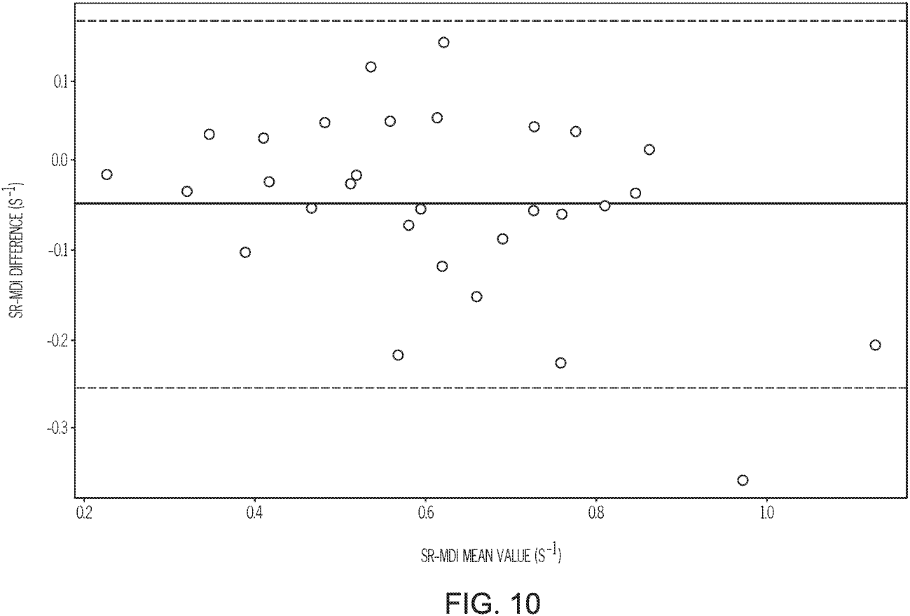

SR-MDI from the three long-axis views showed good intra-observer agreement with intraclass correlation coefficient 0.85 (95% Cl 0.71-0.92) and bias -0.04 s⁻¹ (95% Cl -0.25-0.17) (FIG. 10). Coefficient of variability of SR-MDI was 18.1%.

### Conclusion

The results have shown that the present method may be used for detecting cardiac dyssynchrony. Furthermore, there is a correlation between the mechanical dyssynchrony index calculated using this method and the response to CRT. The results show that dyssynchrony detected with the presently disclosed method is associated with functional improvement after CRT. The elaborate analysis of the cardiac deformation provides a quantitative assessment of the degree of cardiac dyssynchrony of a subject. Thereby, the present method may be used to select subjects that are expected to be responding to CRT and benefit from the treatment.

What is claimed is:

1. A method for at least one of detecting and quantitatively assessing cardiac dyssynchrony of a subject, the method comprising:
determining, from a plurality of medical imaging scans obtained over a time interval representing a preselected part of a cardiac cycle, each scan showing at least part of a myocardium of a heart of the subject and each scan obtained at each time point of the time interval providing a plurality of values of a predefined myocardial deformation parameter of the at least part of the myocardium, at least 60 matrices representing at least a myocardial deformation deviation in at least 60 degrees around an axis passing through a cardiac chamber and an apex of the heart of the subject such that each matrix represents myocardial deformation deviation between opposite parts of the cardiac chamber over the time interval; and
calculating a quantitative assessment of the cardiac dyssynchrony of the subject based on the at least 60 matrices, wherein a plurality of values of each of the at least 60 matrices are used to calculate at least one statistical parameter selected from the group of: standard deviation, variance, mean, harmonic mean, median, mode, range, quantile and maximum value of a histogram,
wherein 2D myocardial deformation parameter values from multiple views of the heart are used to calculate 3D myocardial deformation parameter values of the at least part of the myocardium by interpolating the 2D myocardial deformation parameter values such that the 3D myocardial deformation parameter values are used for calculating the cardiac dyssynchrony of the subject.

2. The method of claim 1, wherein the predefined myocardial deformation parameter is selected from: strain, strain rate, torsion, torsion rate, rotation, rotation rate, twist, twist rate, untwist and untwist rate.

3. The method of claim 1, wherein the plurality of medical imaging scans include one or more of: echocardiography, speckle tracking echocardiography, tissue Doppler echocardiography, feature tracking cardiac magnetic resonance imaging, tagging cardiac magnetic resonance imaging, and cardiovascular magnetic resonance imaging.

4. (canceled)

5. The method of claim 1, wherein the cardiac chamber is selected from: a right atrium, a left atrium, a right ventricle, and a left ventricle.

6. The method of claim 1, wherein the plurality of medical imaging scans represent an apical view.

7. The method of claim 1, wherein the myocardial deformation deviation matrix represents a single plane of the heart and wherein multiple myocardial deformation deviation matrices are determined for multiple planes of the heart, respectively.

8. (canceled)

9. The method of claim 1, wherein the preselected part of the cardiac cycle is selected from: a systolic part of the cardiac cycle or a diastolic part of the cardiac cycle.

10. (canceled)

11. (canceled)

12. (canceled)

13. The method of claim 1, wherein the plurality of medical imaging scans provide a plurality of myocardial deformation parameter values along a cardiac chamber wall of a left ventricle and wherein the plurality of medical imaging scans provide a plurality of myocardial deformation parameter values covering an interventricular septum and the myocardium.

14. The method of claim 1, wherein the opposite parts of the cardiac chamber are opposite walls of the cardiac chamber.

15. The method of claim 1, wherein:
the myocardial deformation deviation for each time point is calculated by mirroring the first half of the plurality of myocardial deformation parameter values and subtracting this from the second half of the plurality of myocardial deformation parameter values, and
the first and second halves of the plurality of myocardial deformation parameter values correspond to the opposite parts of the cardiac chamber.

16. The method of claim 1, wherein the cardiac dyssynchrony of the subject is calculated based on at least 100 myocardial deformation deviation values covering one of the cardiac chambers.

17. (canceled)

18. The method of claim 1, wherein the cardiac dyssynchrony is quantified by:
dispersion of strain rate differences in two opposing walls of at least one cardiac chamber, or
the strain rate-based mechanical dyssynchrony index (SR-MDI) defined as the standard deviation of all strain rate differences between opposing walls of at least one cardiac chamber.

19. A system for at least one of detecting and quantitatively assessing cardiac dyssynchrony of a subject, the system comprising:
a processor; and
a non-transitory, processor-readable storage medium containing one or more programming instructions thereon that, when executed, causes the processor to:
determine, from a plurality of medical imaging scans obtained over a time interval representing a preselected part of a cardiac cycle, each scan showing at least part of a myocardium of a heart of the subject and each scan obtained at each time point of the time interval providing a plurality of values of a predefined myocardial deformation parameter of the at least part of the myocardium, at least 60 matrices representing at least a myocardial deformation deviation in at least 60 degrees around an axis passing through a cardiac chamber and an apex of the heart of the subject such that each matrix represents myocardial deformation deviation between opposite parts of the cardiac chamber over the time interval, and
calculate a quantitative assessment of the cardiac dyssynchrony of the subject based on the at least 60 matrices, wherein a plurality of values of each of the at least 60 matrices are used to calculate at least one statistical parameter selected from the group of: standard deviation, variance, mean, harmonic mean, median, mode, range, quantile and maximum value of a histogram,
wherein 2D myocardial deformation parameter values from multiple views of the heart are used to calculate 3D myocardial deformation parameter values of the at least part of the myocardium by interpolating the 2D myocardial deformation parameter values such that the 3D myocardial deformation parameter values are used for calculating the cardiac dyssynchrony of the subject.

20. A system for at least one of detecting and quantitatively assessing cardiac dyssynchrony of a subject, the system comprising
a medical imaging device that acquires at least one medical imaging scan of at least part of a myocardium of a heart of the subject, the at least one medical imaging scan providing a plurality of values of a predefined myocardial deformation parameter of the at least part of the myocardium; and
a processing unit configured to:
determine, from a plurality of medical imaging scans obtained over a time interval representing a preselected part of a cardiac cycle, each scan showing at least part of the myocardium of the heart of the subject and each scan obtained at each time point of the time interval providing a plurality of values of a predefined myocardial deformation parameter of the at least part of the myocardium, at least 60 matrices representing at least a myocardial deformation deviation in at least 60 degrees around an axis passing through a cardiac chamber and an apex of the heart of the subject such that each matrix represents myocardial deformation deviation between opposite parts of the cardiac chamber over the time interval, and
calculate a quantitative assessment of the cardiac dyssynchrony of the subject based on the at least 60 matrices, wherein a plurality of values of each of the at least 60 matrices are used to calculate at least one statistical parameter selected from the group of: standard deviation, variance, mean, harmonic mean, median, mode, range, quantile and maximum value of a histogram,
wherein 2D myocardial deformation parameter values from multiple views of the heart are used to calculate 3D myocardial deformation parameter values of the at least part of the myocardium by interpolating the 2D myocardial deformation parameter values such that the 3D myocardial deformation parameter values are used for calculating the cardiac dyssynchrony of the subject.

21. (canceled)

22. A method for at least one of detecting and quantitatively assessing cardiac dyssynchrony of a subject, the method comprising:
determining, from at least one medical imaging scan showing at least part of a myocardium of a heart of the subject and providing a plurality of values of a predefined myocardial deformation parameter of the at least part of the myocardium, a myocardial deformation deviation between pairs of myocardial deformation parameter values selected from the myocardium of opposite parts of a cardiac chamber; and
calculating the cardiac dyssynchrony of the subject based on the myocardial deformation deviation,
wherein the plurality of myocardial deformation parameter values are provided by continuously scanning the at least part of the myocardium in 2D from multiple directions over at least part of a systolic part of a cardiac cycle, and wherein the 2D myocardial deformation parameter values are interpolated by means of cubic spline to form 3D myocardial deformation parameter values used for calculating the cardiac dyssynchrony of the subject.

23. A method for at least one of detecting and quantitatively assessing cardiac dyssynchrony of a subject, the method comprising:
determining, from at least one medical imaging scan showing at least part of a myocardium of a heart of the subject and providing a plurality of values of a predefined myocardial deformation parameter of the at least part of the myocardium, a myocardial deformation deviation between pairs of myocardial deformation parameter values selected from the myocardium of opposite parts of a cardiac chamber; and
calculating the cardiac dyssynchrony of the subject based on the myocardial deformation deviation,
wherein the plurality of medical imaging scans are 2D scans obtained for multiple views of the heart and used to calculate at least one 3D image by interpolating the 2D scans, wherein the at least one 3D image provides a plurality of values of the myocardial deformation parameter for the at least part of the myocardium which is used for calculating the cardiac dyssynchrony of the subject.
